(19) United States
(12) Patent Application Publication
(10) Pub. No.: US 2004/0129894 A1
(43) Pub. Date: Jul. 8, 2004

(54) Mercury lamp with electronic ballast and use thereof

(76) Inventors: Marc Coulombe, Drummondville (CA); Steve Morrissette, lle Perrot (CA)

Correspondence Address: BAKER & HOSTETLER LLP, Washington Square, Suite 1100, 1050 Connecticut Avenue, N.W., WASHINGTON, DC, 20036, US

(21) Appl. No.: 10/673,464

(22) Filed: Sep. 30, 2003

Related U.S. Application Data
(60) Provisional application No. 60/414,631, filed on Oct. 1, 2002.

(51) Int. Cl. G01N 21/64

(52) U.S. Cl. 250/461.1

(57) ABSTRACT

The present invention is directed to a combination of a mercury arc lamp placed in a reflector bulb and an electronic ballast to ballast the same. Use of the combination for projecting UVB and UVC radiation is also taught. The combination finds particular application for reptiles and birds that require UVB radiation but low power. The invention can also be used as a germicide, a source of UV for human beings and to treat skin diseases such as psoriasis. It can further be used for curing materials. The combination is advantageously portable, and can be adapted to be clipped on to an object.

### FIELD OF THE INVENTION

The present invention relates to a mercury arc tube, reflector and an electronic ballast, and novel uses therefor.

### DESCRIPTION OF THE PRIOR ART

Self-ballasted mercury arc lamps are well known in the art and have been in use for some time. These lamps are self-ballasted by a filament in the lamp. They were originally used as a more efficient lighting source. However, mercury arc lamps have fallen by the wayside lately given the new lighting sources which are more efficient. One of the more serious drawbacks of the mercury arc lamp is the presence of the incandescent filament which wastes a lot of energy.

One of the by-products of mercury lamps is the generation of UV rays. UV rays are used for a variety of uses. These rays however are presently created by large, bulky mercury fixtures, or in some cases fluorescent tubes. Fluorescent tubes do not generate that much UVB to start with, and whatever they generate cannot be projected, only radiated. At one foot away from the tube, there are negligible amounts of UVB. As for mercury lamps, they are presently cumbersome and non-portable.

Self-ballasted mercury lamps typically produce high wattage and heat, in the order of 150-200 W. The lowest wattage a self-ballasted mercury lamp can attain is approximately 110 W, which is much too hot for some applications, such as in cages for reptiles and birds.

An advantage of self-ballasted mercury lamps is that they can be screwed into any incandescent socket, and depending on the shape of the glass enclosure (R40, Par), the light emitted by the mercury lamp can be projected, as opposed to radiated.

Also known in the art are mercury lamps with magnetic ballasts, which are used for general lighting purposes. However, such magnetic ballasts are generally part of fixtures or mounted separately and are bulky. The complete unit itself is not portable.

### SUMMARY OF THE INVENTION

It is an object of the present invention to provide a mercury lamp which outputs less power than self-ballasted mercury lamps with the same flexibility of use. It is also an object of the present invention to use such a low-power mercury lamps for uses other than lighting.

In accordance with the invention, these and other objects are achieved with a mercury lamp ballasted with an electronic ballast.

More specifically, a first object of the invention is to provide in combination, a mercury arc lamp including an arc tube, said arc tube being mounted in a reflector bulb adapted to be screwed into a standard light bulb socket and an electronic ballast for ballasting said mercury arc lamp.

A second object of the invention is to provide a method for projecting UV radiation comprising the steps of:

(a) providing a reflector with a mercury arc tube, said reflector having a base screwed into a standard light socket holder and an opposite end; and

(b) providing an electronic ballast for ballasting said mercury arc lamp;

wherein when said lighting said mercury arc tube is lit, UV radiation produced by said lit mercury arc tube is projected out of said opposite end.

A third object of the invention is to provide a method for providing UVB radiation inside an enclosure, said method including the steps of:

(a) providing a reflector with a mercury arc tube, said reflector having a base screwed into a standard light socket holder and an opposite end;

(b) placing said reflector proximate said enclosure; and

(c) providing an electronic ballast for ballasting said mercury arc lamp;

wherein when said mercury arc tube is lit, UVB radiation produced by said mercury arc tube is projected out of said opposite end.

A fourth object of the invention is to provide a method for projecting UVC radiation comprising the steps of:

(a) providing a reflector with a mercury arc tube, said reflector having a base screwed into a standard light socket holder and an opposite, open end; and

(b) providing an electronic ballast for ballasting said mercury arc tube;

wherein when said mercury arc tube is lit, UVC radiation produced by said mercury arc tube is projected out of said opposite, open end.

A fifth object of the invention is to provide a method for projecting UV radiation for treating skin disorders or for assisting in the production of vitamin D, comprising the steps of:

(a) providing a reflector with a mercury arc tube, said reflector having a base screwed into a standard light socket holder and an opposite end;

(b) providing an electronic ballast for ballasting said mercury arc tube;

wherein when said mercury arc tube is lit, UV radiation produced by said mercury arc tube is projected out of said opposite end.

A sixth object of the invention is to provide a method for projecting UV radiation for use as a germicide, comprising the steps of:

(a) providing a reflector with a mercury arc tube, said reflector having a base screwed into a standard light socket holder and an opposite end;

(b) providing an electronic ballast for ballasting said mercury arc tube;

wherein when said mercury arc tube is lit, UV radiation produced by said mercury arc tube is projected out of said opposite end.

Finally, a seventh object of the invention is to provide a method for projecting UV radiation for curing materials, comprising the steps of:

(a) providing a reflector with a mercury arc tube, said reflector having a base screwed into a standard light socket holder and an opposite end;

(b) providing an electronic ballast for ballasting said mercury arc tube;

wherein when said mercury arc tube is lit, UV radiation produced by said mercury arc tube is projected out of said opposite end.

### BRIEF DESCRIPTION OF THE DRAWINGS

The present invention and its advantages will be more easily understood after reading the following non-restrictive description of preferred embodiments thereof, made with reference to the following drawings in which:

FIG. 1 is a schematic representation of a Par38 60 watt UV flood lamp, a Par30 60 watt UV lamp, a Par30 60 watt two-piece UV lamp and a R40 60 watt UV flood lamp, going from left to right;

FIG. 2 is a flow chart of an electronic ballast for a mercury arc tube inserted into a bulb as shown in FIG. 1, according to a preferred embodiment of the invention;

FIG. 3 is a schematic representation of a desk lamp in which the mercury lamp for UV can be inserted, including a timer and electronic ballast within the base, according to a preferred embodiment of the invention; and

FIG. 4 is a schematic representation of a holder with a clip-on attachment, which is plugged into the electronic ballast, which is in turn plugged into a socket.

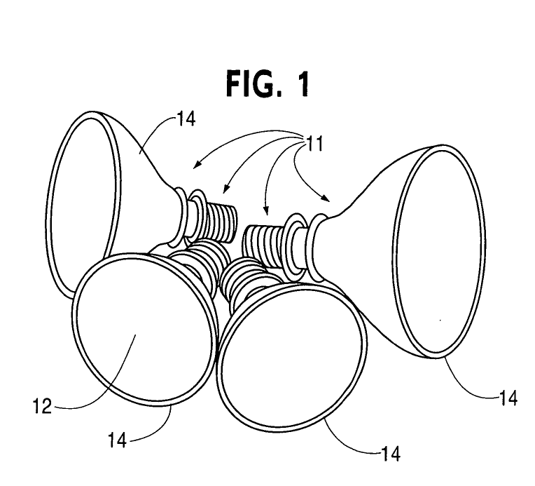

### DESCRIPTION OF A PREFERRED EMBODIMENT OF THE INVENTION

Reptiles and birds that live in tropical climates require UVB to create vitamin D. Without sufficient amounts of vitamin D, these animals can develop various bone diseases.

As mentioned previously, fluorescent tubes and mercury self-ballasted lamps (100 W, 160 W and 250 W) are presently being used for this purpose.

However, there is a need for a lower wattage lamp for use in the reptile and bird trade, in order to accommodate smaller cages and birds, which cannot support the heat generated by a 100 W or more lamp.

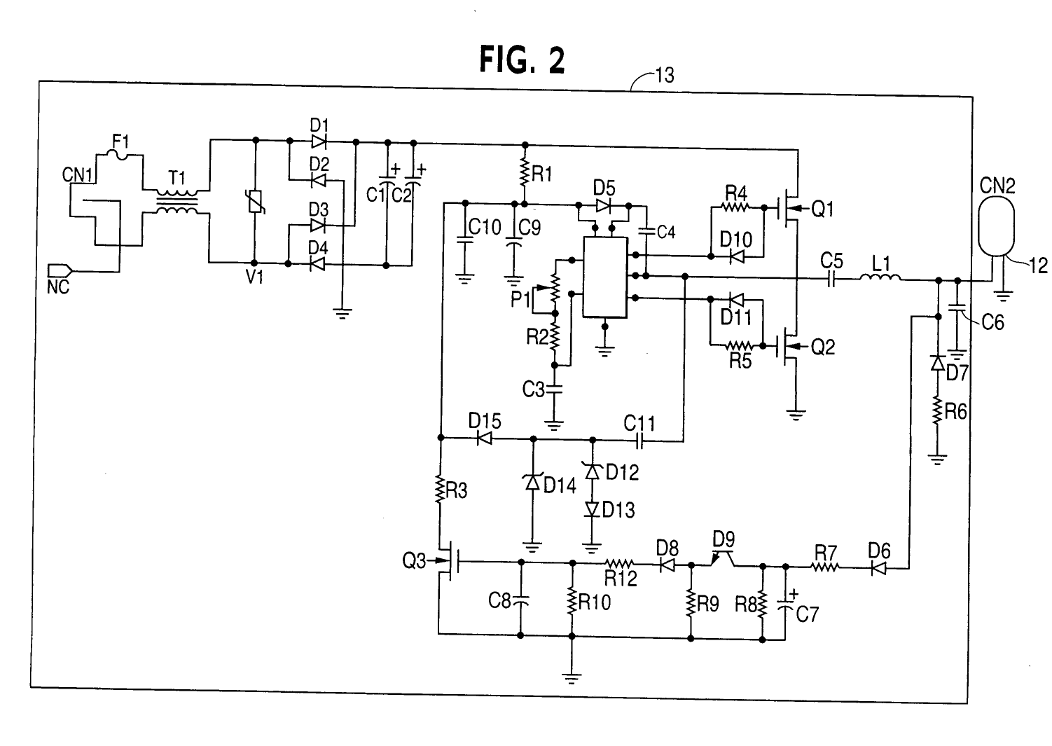

An aspect of the invention is thus to ballast the mercury lamp 11 with an electronic ballast 13, in order to lower the wattage and heat of the lamp. An electronic ballast 13 for this purpose is shown in FIG. 2, but it should be understood that other configurations for the electronic ballast can be used without departing from the scope of the present invention. The ballast of FIG. 2 is a specially designed 75 kHz ballast which provides the necessary voltage for lighting the lamp, and the necessary current to maintain it lit.

The mercury lamp 11 is comprised of a mercury arc tube 12 which is mounted inside a reflector bulb 14, which is provided with a base adapted to be screwed into a standard light socket holder 15.

Such a combination of a mercury lamp 11 and an electronic ballast 13 to provide UVB for reptiles and birds is one aspect of the present invention. In one embodiment, as shown in FIG. 4, holder 15 receives the arc lamp 11, and is placed proximate to a cage for projecting UV radiation inside the cage. The holder 15 is connected to the electronic ballast 13 with a standard cord and plug and the ballast 13 is in turn plugged into an electrical outlet. In this case, the holder is further preferably provided with a clip-on attachment 16.

Similarly, vitamin D is essential in humans to maintain a healthy skeleton. Many elderly people, and those who live in cold climates are not exposed to sufficient sunlight, and thus some have vitamin D deficiencies, leading to bone loss and fractures.

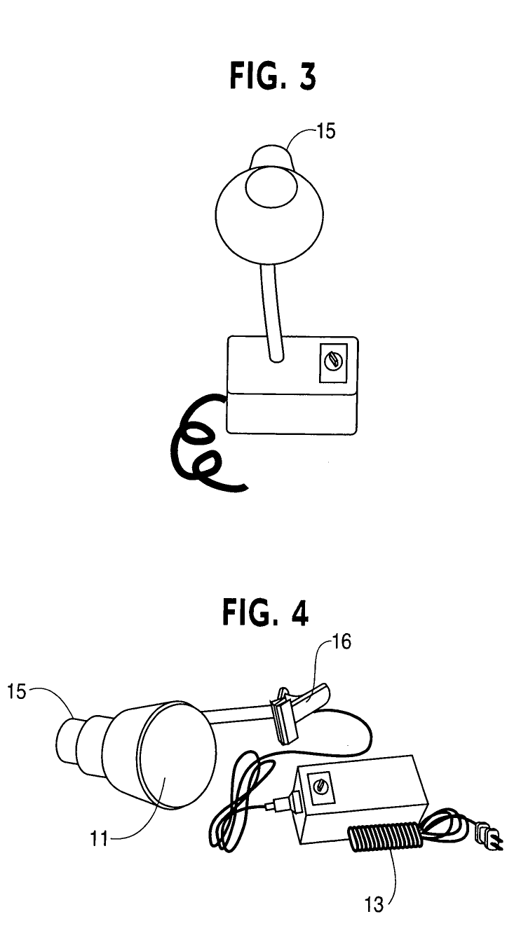

Most sources of UVB for humans are either fluorescent lamps that require very close proximity or bulky sun lamps, which are very hot. Accordingly, it is another aspect of the present invention to provide a small 60 W mercury lamp with an electronic ballast to produce UVB in a more useful package. The arc tube is preferably mounted in a reflector bulb, such as R40 or Par38, which can be inserted in an ordinary medium lamp socket. The lamp and ballast can also be integrated into a desk lamp configuration, as shown in FIG. 3, including a timer or provided with a holder which can be screwed into a socket or clamped onto a surface. The ballast itself can be plugged into a wall socket or put on a shelf. Since the lamp projects a beam of light, and draws only 60 W, which is considerably lower than other sources, it is more comfortable to sit under. Furthermore, it is cooler, and because of the projection of the beam, it can be used at a distance. Such a configuration is also highly portable.

It is also known that skin diseases such as psoriasis respond to exposure to UVA and UVB. Consequently, it is also an object of the present invention to provide a mercury lamp with an electronic ballast to treat some forms of skin diseases.

Another use for the lamp of the present invention is the generation of UVC. UVC, or short wave radiation, is an effective germicidal for microorganisms such as bacteria, bacterial spores, moulds, yeasts, protozoa and viruses. Mercury lamps are a primary source of UVC, and typically take the form of a tube (resembling a fluorescent tube), requiring a fixture and a ballast.

An object of the invention is the use of a low power, electronically ballasted mercury arc tube to produce UVC. The ballast can be plugged into an outlet, put on a shelf or sit on a desk lamp fixture. The arc tube is inserted into a two-piece reflector glass bulb with the front pane of glass removed (since glass absorbs UVC). The bulb can be screwed into any medium based socket. The bulb has an aluminium reflector which directs the UVC beam generated by the arc tube. The result is a portable, lightweight UVC source which projects a beam of UVC at a distance.

The invention also covers other uses of the combination of a mercury lamp with an electronic ballast, where such uses require UV light, low wattage and portability. For example, UV radiation is also sometimes used for curing materials. Consequently, the invention can be used for spot curing materials, or for curing materials where lamp portability is important or the circumstances do not require investing heavily in an elaborate set-up for curing materials, such as for small runs.

The present invention thus offers a small, lightweight, portable device for the generation of UV radiation. The reader will appreciate that elements such as an on/off switch, electrical supply and the like need not be further detailed herein.

Although the present invention has been explained hereinabove by way of a preferred embodiment thereof, it should be pointed out that any modifications to this preferred embodiment within the scope of the appended claims is not deemed to alter or change the nature and scope of the present invention.

1. In combination, a mercury arc lamp including an arc tube, said arc tube being mounted in a reflector bulb adapted to be screwed into a standard light bulb socket and an electronic ballast for ballasting said mercury arc lamp.

2. The combination of claim 1, wherein said mercury arc lamp is adapted to consume 60 W of power or less.

3. The combination of claim 1, wherein said standard light bulb socket is further provided with a pinch clamp fixture for clamping said socket to an object, said fixture being connected to said electronic ballast.

4. The combination of claim 1, wherein said electronic ballast is housed in a fixture and said fixture is integral to said socket so that said combination is adapted to be placed on a desk.

5. The combination of claim 4, wherein said housing further includes a timer operatively connected to said electronic ballast.

6. A method for projecting UV radiation comprising the steps of:
(c) providing a reflector with a mercury arc tube, said reflector having a base screwed into a standard light socket holder and an opposite end; and
(d) providing an electronic ballast for ballasting said mercury arc lamp;
wherein when said lighting said mercury arc tube is lit, UV radiation produced by said lit mercury arc tube is projected out of said opposite end.

7. A method for providing UVB radiation inside an enclosure, said method including the steps of:
(d) providing a reflector with a mercury arc tube, said reflector having a base screwed into a standard light socket holder and an opposite end;
(e) placing said reflector proximate said enclosure; and
(f) providing an electronic ballast for ballasting said mercury arc lamp;
wherein when said mercury arc tube is lit, UVB radiation produced by said mercury arc tube is projected out of said opposite end.

8. A method for projecting UVC radiation comprising the steps of:
(c) providing a reflector with a mercury arc tube, said reflector having a base screwed into a standard light socket holder and an opposite, open end; and
(d) providing an electronic ballast for ballasting said mercury arc tube;
wherein when said mercury arc tube is lit, UVC radiation produced by said mercury arc tube is projected out of said opposite, open end.

9. A method for projecting UV radiation for treating skin disorders or for assisting in the production of vitamin D, comprising the steps of:
(c) providing a reflector with a mercury arc tube, said reflector having a base screwed into a standard light socket holder and an opposite end;
(d) providing an electronic ballast for ballasting said mercury arc tube;
wherein when said mercury arc tube is lit, UV radiation produced by said mercury arc tube is projected out of said opposite end.

10. A method for projecting UV radiation for use as a germicide, comprising the steps of:
(c) providing a reflector with a mercury arc tube, said reflector having a base screwed into a standard light socket holder and an opposite end;
(d) providing an electronic ballast for ballasting said mercury arc tube;
wherein when said mercury arc tube is lit, UV radiation produced by said mercury arc tube is projected out of said opposite end.

11. A method for projecting UV radiation for curing materials, comprising the steps of:
(c) providing a reflector with a mercury arc tube, said reflector having a base screwed into a standard light socket holder and an opposite end;
(d) providing an electronic ballast for ballasting said mercury arc tube;
wherein when said mercury arc tube is lit, UV radiation produced by said mercury arc tube is projected out of said opposite end.
